(19) United States
(12) Patent Application Publication
(10) Pub. No.: US 2026/0158046 A1
(43) Pub. Date: Jun. 11, 2026

(54) TREATMENT OF NEURODEGENERATIVE DISORDERS WITH DOXYCYCLINE ALONE OR IN COMBINATION WITH LEVODOPA

(71) Applicant: Biohaven Therapeutics Ltd., New Haven, CT (US)

(72) Inventor: Vladimir CORIC, Madison, CT (US)

(21) Appl. No.: 19/152,064

(22) PCT Filed: Feb. 6, 2024
(86) PCT No.: PCT/US2024/014540

§ 371 Date: Jul. 30, 2025

Related U.S. Application Data
(60) Provisional application No. 63/483,397, filed on Feb. 6, 2023.

(51) Int. Cl. A61K 31/65; A61K 31/198; A61K 45/06; A61P 1/00; A61P 25/16

(52) U.S. Cl. CPC A61K 31/65; A61K 31/198; A61K 45/06; A61P 1/00; A61P 25/16

(57) ABSTRACT

Provided herein are methods of treating a neurodegenerative disorder (e.g., Parkinson's Disease) or a symptom thereof in a patient, by administering to the patient doxycycline (or a pharmaceutically acceptable salt thereof) either alone or in combination with other therapeutic agents (e.g., levodopa).

### CROSS-REFERENCE TO RELATED APPLICATION

This application claims priority to, and the benefit of, U.S. Provisional Application No. 63/484,286 (filed Feb. 10, 2023). The entire content of the aforementioned application is incorporated herein by reference.

### BACKGROUND

Neurodegenerative disorders affect millions of people worldwide and are primarily characterized by neuron loss. The most common neurodegenerative disorders are Alzheimer's and Parkinson's disease (PD). Although there are several medicines currently approved for managing neurodegenerative disorders, most of them only help with associated symptoms. Moreover, while there has been some therapeutic success with surgeries and other techniques, clinical acceptance of these approaches is limited due to varying concerns about their long-term benefit, owing to the potential damage to the brain barrier (see, e.g., Lamptey RNL, et al., “A Review of the Common Neurodegenerative Disorders: Current Therapeutic Approaches and the Potential Role of Nanotherapeutics”, Int. J. Mol. Sci. 2022 Feb. 6; 23 (3): 1851). The lack of pathogenesis-targeting therapies is primarily due to the restrictive effects of the blood-brain barrier (BBB), which keeps close to 99% of all “foreign substances” out of the brain. The advanced nature of the BBB, combined with the poor permeative potency of most, if not all drugs, accounts for the lack of suitable treatment options for neurodegenerative disorders.

Accordingly, it is an object of the present disclosure to provide improved methods for treating patients with Neurodegenerative disorders and, in particular, PD.

### SUMMARY OF THE INVENTION

Provided herein are methods for treating various neurodegenerative disorders or symptoms by administering doxycycline (or a pharmaceutically acceptable salt thereof) either alone or in combination with other therapeutic agents (e.g., levodopa) to a patient in need thereof.

Exemplary neurodegenerative disorders include, but are not limited to, amyotrophic lateral sclerosis (ALS), Huntington's disease, Parkinson's Disease (PD), supranuclear palsy, frontotemporal dementia, frontotemporal lobar degeneration, frontotemporal neurocognitive disorder, Alzheimer's disease, mild cognitive impairment, mild cognitive impairment due to Alzheimer's disease, ataxia, motor neuron disease, multiple system atrophy, hereditary ataxia, dementia, memory loss, mental retardation, Rett Syndrome, progressive supranuclear palsy, Creutzfeldt-Jakob disease, neurofibromatosis, brain injury, stroke, multiple sclerosis, and α-synucleinopathy (e.g., Lewy body disease). In one embodiment, the neurodegenerative disorder is Alzheimer's disease. In another embodiment, the neurodegenerative disorder is PD.

Exemplary symptoms include, but are not limited to abnormal mobility, abnormal balance, abnormal movements (e.g., tremor), abnormal swallowing, abnormal bladder and bowel function (e.g., irritable bowel, belly pain, belly discomfort, diarrhea, abdominal bloating, abdominal gas, abdominal bloating, and/or constipation), blood pressure fluctuation, abnormal sleep, abnormal breathing (e.g., shortness of breath), abnormal heart function (e.g., heart palpitations), abnormal memory, abnormal cognitive abilities, abnormal mood, abnormal speech, anxiety, depression, stress, fatigue, feelings of panic, fear, uneasiness, problems in sleeping, cold or sweaty hands and/or feet, mood liability, mania, impaired concentration or attention, cognitive problems, obsessions, compulsions, repetitive behaviors, aggression, social phobias or impairments, stage fright, an inability to be still and calm, dry mouth, numbness or tingling in the hands or feet, nausea, muscle tension, dizziness apathy, elation, disinhibition, irritability, wandering, and/or combinations thereof.

Doxycycline (or a pharmaceutically acceptable salt thereof) can be administered to a patient by any suitable means. In one embodiment, doxycycline is formulated as a powder for suspension. In another embodiment, doxycycline is formulated as a capsule (e.g., extended release). In another embodiment, doxycycline is formulated as a tablet (e.g., delayed release). In another embodiment, doxycycline is formulated as a syrup.

In some embodiments, doxycycline is administered to the patient in a therapeutically active amount. In some embodiments, doxycycline is administered to the patient at a dose of between 50 mg to 200 mg once a day (e.g., 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg). In one embodiment, doxycycline is administered at a dose of 50 mg once a day. In another embodiment, doxycycline is administered at a dose of 100 mg once a day. In another embodiment, the patient is an adult patient and doxycycline is administered at a dose of 100 mg every 12 hours on the first day, then 100 mg once a day or 50 to 100 mg (e.g., 50 mg, 55 mg, 60 mg, 65 mg, 70 mg, 75 mg, 80 mg, 85 mg, 90 mg, 95 mg, 100 mg) every 12 hours. In another embodiment, doxycycline is administered to the patient at subtherapeutic dose.

In some embodiments, the patient has previously been administered levodopa (or a pharmaceutically acceptable salt thereof). For example, in some embodiments, the patient has been treated with levodopa for 3 months, 6 months, 9 months, 1 year, 2 years, 3 years, 4 years or more prior to being treated according to the methods described herein.

Also provided herein are methods of treating a neurodegenerative disorder (e.g., PD) or symptom (e.g., tremor) in a patient, by administering to the patient a therapeutically active amount of doxycycline (or a pharmaceutically acceptable salt thereof) in combination with a therapeutically active amount of levodopa (or a pharmaceutically acceptable salt thereof). In one embodiment, doxycycline and levodopa are administered concurrently. In another embodiment, doxycycline and levodopa are administered sequentially in time. For example, in one embodiment, levodopa is administered prior to administration of doxycycline. In another embodiment, doxycycline is administered prior to administration of levodopa.

Levodopa (or a pharmaceutically acceptable salt thereof) can be administered to a patient by any suitable means. In one embodiment, levodopa is formulated as an oral tablet or capsule. In another embodiment, levodopa is formulated as an oral inhalation. In another embodiment, levodopa is formulated as an infusion.

In some embodiments, levodopa (or a pharmaceutically acceptable salt thereof) is administered to the patient in a therapeutically active amount. In some embodiments, levodopa is administered in combination (e.g., separately or in a single formulation) with the drug carbidopa, which reduces or prevents the nausea that levodopa alone can cause.

In one embodiment, levodopa is administered at a total dose of 100 to 1,000 mg of levodopa per day (e.g., administered as a divided dose, 3 to 4 times a day, as an immediate release tablet). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg.

In another embodiment, levodopa is administered at a total daily dose of 100 to 1,600 mg (e.g., administered in divided doses as a controlled-release tablet). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg. In a particular embodiment, levodopa is administered at a dose of 100 mg once per day.

In another embodiment, levodopa is administered at a total daily dose of 100 to 1,000 mg (e.g., administered as a divided dose, 3 to 4 times a day, as an orally disintegrating tablet).

In another embodiment, levodopa is administered at a dose of up to 2,000 mg over 16 hours (e.g., as an enteral suspension). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg. In a particular embodiment, levodopa is administered at a dose of 100 mg once per day.

In another embodiment, levodopa is administered at a total daily dose of 855 to 2,340 mg of levodopa (e.g., administered in divided as an extended release capsule).

In another embodiment, levodopa is administered at a total daily dose of 100 to 1,600 mg (e.g., administered as a tablet). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In another embodiment, levodopa is administered at a dose of 42 to 84 mg, up to 5 times per day (e.g., as a capsule of inhalation powder). For example, levodopa can be administered at a dose of 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, or 84 mg. In one embodiment, levopoda is administered once a day, twice a day, three times a day, four times a day, or five times a day.

In another embodiment, levodopa is administered to the patient at subtherapeutic dose.

Also provided herein are methods of treating a neurodegenerative disorder (e.g., PD) or symptom (e.g., tremor) in a patient, by administering to the patient a therapeutically active amount of doxycycline (or a pharmaceutically acceptable salt thereof) in combination with a therapeutically active amount of levodopa (or a pharmaceutically acceptable salt thereof), wherein administration of doxycycline and levodopa in combination results in a synergistic therapeutic effect compared to administration of either doxycycline or levodopa alone. to administration of either doxycycline or levodopa alone.

Further provided are methods of treating a neurodegenerative disorder (e.g., PD) or symptom (e.g., tremor) in a patient by administering doxycycline (or a pharmaceutically acceptable salt thereof) in combination with levodopa, wherein what would normally be a subtherapeutic dose of doxycycline or levodopa alone, is rendered effective by the combination. Thus, a dose of doxycycline below the standard therapeutic dose and/or a dose of levodopa below the standard therapeutic dose, which would ordinarily be without therapeutic effect in humans, is found to have therapeutic effective when doxycycline and levodopa are administered in combination. In other words, by administering a combination of doxycycline and levodopa, at least one or both of doxycycline and/or levodopa can be at administered at a subtherapeutic dose, while still resulting in a similar efficacy.

Accordingly, in on aspect, methods of treating a neurodegenerative disorder (e.g., PD) or symptom (e.g., tremor) in a patient are provided, which comprise administering to the patient doxycycline (or a pharmaceutically acceptable salt thereof) in combination with levodopa (or a pharmaceutically acceptable salt thereof), wherein at least one of doxycycline and/or levodopa is administered at a subtherapeutic dose. In some embodiments, the subtherapeutic dose of doxycycline and/or subtherapeutic dose of levodopa is a dose which is lower than the dose of doxycycline and/or the dose of levodopa, required to obtain a therapeutic effect in the patient, when administered alone.

The efficacy of the treatment methods provided herein can be assessed using any suitable means. In one embodiment, the neurodegenerative disorder is PD and the treatment results in an improvement according to a recognized clinical scale for evaluating PD. For example, in one embodiment, the neurodegenerative disorder is PD and the treatment results in an improvement according to the Unified Parkinson Disease Rating Scale (UPDRS). A score of 199 on the UPDRS scale represents the worst (total disability) with a score of zero representing (no disability). Accordingly, in one embodiment, the treatment results in a decrease in score compared to baseline by, for example, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 199 points. In another embodiment, the treatment results in a 5, 10, 15, 20, 25, 30, 35, 40, 45, 50, 55, 60, 65, 70, 75, 80, 85, 90, 95, 100% decrease in score compared to baseline.

In another embodiment, the neurodegenerative disorder is PD and the treatment results in an improvement according to the Hoehn and Yahr Staging Scale. The Hoehn and Yahr Staging Scale includes 7 different stages ranging from no disease (Stage 1) to wheelchair bound or bedridden (Stage 5), i.e., Stage 0, Stage 1, Stage 1.5, Stage 2, Stage 2, Stage 2.5, Stage 3, Stage 4, and Stage 5. In one embodiment, the treatment results in a one or more stage decrease in score compared to baseline (e.g., a one, two, three, four, five, six, seven, or eight stage decrease).

In another embodiment, the neurodegenerative disorder is PD and the treatment results in an improvement according to the Schwab and England Rating of Activities of Daily Living. This rating varies from 0 (completely independent) to 100% (vegetative functions), using 10% increments and includes 11 possible ratings, i.e., 0%, 10%, 20%, 30%, 40%, 50%, 60%, 70%, 80%, 90%, and 100%. In one embodiment, the treatment results in a one or more rating decrease compared to baseline (e.g., a one, two, three, four, five, six, seven, eight, one or ten rating decrease).

In some embodiments, the neurodegenerative disorder is PD and the treatment results in an improvement in tremor (e.g., a reduction in tremor), for example, by about 5%, 10%, 15%, 20%, 25%, 30%, 35%, 40%, 45%, 50%, 55%, 60%, 65%, 70%, 75%, 80%, 85%, 90%, 95%, or 100%). The improvement can be assessed by any suitable means, including, but not limited to electromyography (EMG), an electromagnetic motion tracker, noncontact measurements obtained from devices (e.g., Kinect), a laser Doppler vibrometer, an accelerometer, a gyroscope, or a smart wearable technology device.

Doxycycline (or a pharmaceutically acceptable salt thereof) can be administered according to the methods described herein: (1) alone, (2) in combination with levodopa (or a pharmaceutically acceptable salt thereof), (3) in combination with one or more other therapeutic agents, or (4) in combination with levodopa and one or more other therapeutic agents.

Any other suitable therapeutic agent can be administered. In one embodiment, the other therapeutic agent is a Kv7 channel activator (e.g., retigabine, flupirtine, or endocannabinoid-like molecules).

In another embodiment, the other therapeutic agent is an antibody to alpha-synuclein (e.g., Abeam's “Syn204”, Alexis's “15G 7”, Becton Dickinson's “42”, Chemicon's “7B 2, 12”, NovaCastra's “KM51”, SantaCruz's “Syn211”, or Zymed's “LB509”).

In another embodiment, the other therapeutic agent is a decarboxylase inhibitor, such as carbidopa.

In another embodiment, the other therapeutic agent is a dopamine agonist (e.g., Parlodel (BROMOCRIPTINE®), Cabergoline, Apokyn (APOMORPHINE®), Mirapex (PRAMIPEXOLE®), Requip (ROPINIROLE®), or Neupro (ROTIGOTINE®).

In another embodiment, the other therapeutic agent is a monoamine oxidase (MAO) B inhibitor (e.g., socarboxazid (MARPLAN®), Phenelzine (NARDIL®), Selegiline (EMSAM®), Tranylcypromine (PARNATE®), selegiline hydrochloride (ELDEPRYL®), selegiline hydrochloride (ZELAPAR®), rasagiline (AZILECT®) or safinamide (XADAGO®).

In another embodiment, the other therapeutic agent is a catechol O-methyltransferase (COMT) inhibitor (e.g., entacapone (COMTAN®, COMTESS®, STALEVO®), nebicapone, nitecapone, opicapone (ONGENTYS®), or tolcapone (TASMAR®).

In another embodiment, the other therapeutic agent is an anti-cholinergic agent (e.g., (1) an antimuscarinic agent, such as an antipsychotic (CLOZAPINE®, QUETIAPINE®), atropine, benztropine (COGENTIN®), biperiden, chlorpheniramine, certain SSRIs (PAROXETINE®), dicyclomine (DICYCLOVERINE®), dimenhydrinate, diphenhydramine, doxepin, doxylamine, flavoxate, glycopyrronium/-late, hyoscyamine, ipratropium, orphenadrine, oxitropium, oxybutynin, promethazine, propantheline bromide, scopolamine, solifenacin, tolterodine, tiotropium, tricyclic antidepressants, trihexyphenidyl, amantadine, tropicamide, and umeclidinium, or (2) an antinicotinic agent, such as bupropion, dextromethorphan, doxacurium, dexamethonium, mecamylamine, and tubocurarine).

In another embodiment, the other therapeutic agent is an adenosine receptor antagonist (A2A receptor antagonist) (e.g., caffeine, theophylline or istradefylline (NOURIANZ®), or Nuplazid (PIMAVANSERIN®)).

In another embodiment, the other therapeutic agent is a TNF-alpha targeting agent or inhibitor (e.g., liximab (REMICADE®), adalimumab (HUMIRA®), certolizumab pegol (CIMZIA®), golimumab (SIMPONI®), and etanercept (ENBREL®).

In another embodiment, the other therapeutic agent is a IL17A targeting agent or inhibitor (e.g., Secukinumab (COSENTYX®), Ixekizumab (TALTZ®), Bimekizumab (BIMZELX®), or Brodalumab (SILIQ®).

In another embodiment, the other therapeutic agent is deep brain stimulation.

Further provided are kits that include a dose of doxycycline adapted for use in the methods described herein, e.g., for effective treatment of a neurodegenerative disorder (e.g., PD) or symptom (e.g., tremor). In one embodiment, the kit comprises: (a) a dose of doxycycline and (b) instructions for using doxycycline in the methods described herein. In another embodiment, the kit comprises (a) a dose of doxycycline (b) a dose of levodopa and (c) instructions for using doxycycline and levodopa in the methods described herein. In one embodiment, the dose of doxycycline is a therapeutically effective amount. In one embodiment, the dose of levodopa is a therapeutically effective amount. In one embodiment, the dose of doxycycline and/or levodopa is a subtherapeutic dose.

Also provided are uses of doxycycline for treatment of a neurodegenerative disorder (e.g., PD) or symptom (e.g., tremor), wherein doxycycline (or a pharmaceutically acceptable salt thereof) is administered to the patient in an amount and with a frequency to treat the neurodegenerative disorder or symptom. Further provided are uses of doxycycline in combination with levodopa (or a pharmaceutically acceptable salt thereof) for treatment of a neurodegenerative disorder (e.g., PD) or symptom (e.g., tremor), wherein doxycycline and levodopa are administered to the patient in an amount and with a frequency to treat the neurodegenerative disorder or symptom. In one embodiment, the combination of doxycycline and levodopa results in a synergistic therapeutic effect compared to administration of either doxycycline or levodopa alone. In another embodiment, the combination of doxycycline and levodopa allows for doxycycline and/or levodopa to be administered at a subtherapeutic dose.

### DETAILED DESCRIPTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description.

### I. Definitions

As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

### II. Neurodegenerative Disorders and Symptoms

Neurons are central to the proper functioning of the human brain, as they play a critical role in communication (see, e.g., Lamptey RNL, et al., 2022; and Van den Heuvel M. P., et al., “Network hubs in the human brain”, Trends Cogn. Sci. 2013; 17:683-696). While most neurons originate in the brain, they are present everywhere in the body. During childhood, neural stem cells produce the majority of neurons, the number of which is significantly reduced in adulthood (see, e.g., Ganat Y. M., et al., “Early postnatal astroglial cells produce multilineage precursors and neural stem cells in vivo,” J. Neurosci. 2006; 26:8609-862). Neurons are not immortal. However, the progressive loss of neurons, neuron structure, and/or their functions (known as “neurodegeneration”), is central to the pathophysiology of several brain disorders and is also a major health concern (see, e.g., Przedborski S., et al., “Series Introduction: Neurodegeneration: What is it and where are we?”, J. Clin. Investig. 2003; 111:3-10). Neurodegeneration is associated with dysfunction of the synapse, neural network, and the deposition of physiochemically altered variants of proteins in the brain Diseases with neurodegeneration as their hallmark feature are collectively termed NDs (see, e.g., Lamptey RNL, et al., 2022).

Any neurodegenerative disorder or symptom can be treated according to the methods described herein. Exemplary neurodegenerative disorders include, but are not limited to, amyotrophic lateral sclerosis (ALS), Huntington's disease, PD, supranuclear palsy, frontotemporal dementia, frontotemporal lobar degeneration, Alzheimer's disease, mild cognitive impairment, mild cognitive impairment due to Alzheimer's disease, ataxia, hereditary ataxia, dementia, memory loss, mental retardation, and Rett Syndrome.

The methods described herein can also be used to treat one or more symptoms of a neurodegenerative disorder. Exemplary neurodegenerative disorders include, but are not limited to, anxiety, depression, stress, fatigue, feelings of panic, fear, uneasiness, problems in sleeping, cold or sweaty hands and/or feet, mood liability, mania, impaired concentration or attention, cognitive problems, obsessions, compulsions, repetitive behaviors, aggression, social phobias or impairments, stage fright, shortness of breath, heart palpitations, an inability to be still and calm, dry mouth, numbness or tingling in the hands or feet, nausea, muscle tension, dizziness apathy, elation, disinhibition, irritability, wandering, irritable bowel, belly pain, belly discomfort, diarrhea, change in bowel habits, abdominal bloating, abdominal gas, abdominal bloating, and/or constipation.

### A. Parkinson's Disease

PD is a neurodegenerative disease that typically begins about age 60 and causes slowness of movement (bradykinesia), muscular stiffness (rigidity), tremor, poor postural stability, soft voice, shuffling gait, sudden cessation of movement called freezing, and a paucity of spontaneous movements (akinesia) (see, e.g., Fahn S., “Description of Parkinson's disease as a clinical syndrome,” Ann. N.Y. Acad. Sci. 2003; 991:1-14). Motor manifestations typically begin on one side of the body, only later affecting the other side as well (see, e.g., Rajput A H, et al., “Accuracy of clinical diagnosis in parkinsonism—A prospective study,” Can. J. Neurol. Sci. 1991; 18:275-278). Underlying degeneration of dopaminergic nigrostriatal neurons with subsequent striatal dopamine deficiency forms the basis for pharmacotherapy (see, e.g., McColl C D, et al., “Motor response to levodopa and the evolution of motor fluctuations in the first decade of treatment of Parkinson's disease,” Mov. Disord. 2002; 17:1227-1234).

As used herein, the term “tremor” refers to the involuntary oscillatory and rhythmic movement produced by synchronous or alternating contractions of agonist/antagonistic muscles (see, e.g., Sigcha L, et al., “Automatic Resting Tremor Assessment in Parkinson's Disease Using Smartwatches and Multitask Convolutional Neural Networks”, Sensors (Basel). 2021 Jan. 4; 21 (1): 291. Tremor can be experienced in the hands, head, trunk, or legs (see e.g., Puschmann A., et al., “Diagnosis and Treatment of Common Forms of Tremor”, Semin. Neurol. 2011; 31:65-77). In PD, tremors can appear in the early stages of the disease and reduce the quality of life by interrupting activities such as reading, writing, and eating (see e.g., Hssayeni M. D., et al., “Wearable Sensors for Estimation of Parkinsonian Tremor Severity during Free Body Movements,” Sensors. 2019; 19:4215). Greater than 70% of all PD patients experience resting tremors in the course of the disease and the effects of the tremors tend to be more severe with aging (see, e.g., Baumann C. R., “Epidemiology, diagnosis and differential diagnosis in Parkinson's disease tremor,” Parkinsonism Relat. Disord. 2012; 18: S90-S92. Tremor in PD can be divided into (1) resting tremor, which occurs when patients relax their muscles and (2) action tremor (postural and kinetic), which occurs while the subjects make voluntary muscle movements (see, e.g., Bhidayasiri R., “Differential Diagnosis of Common Tremor Syndromes,” Postgrad. Med. J. 2005; 81:756-762.

Exemplary techniques for assessing PD tremors include, but are not limited to, sensor technologies, such as electromyography (EMG) (see, e.g., Mailankody P., et al., Re-emergent tremor in Parkinson's disease: A clinical and electromyographic study”, J. Neurol. Sci. 2016; 366:33-36; Palmes P., et al., “Pattern Mining of Multichannel sEMG for Tremor Classification”, IEEE Trans. Biomed. Eng. 2010; 57:2795-2805; and Kwon K., et al., “Comparison of motor and non-motor features between essential tremor and tremor dominant Parkinson's disease,” J. Neurol. Sci. 2016; 361:34-38), electromagnetic motion trackers (see, e.g., Perera T., et al., “Clinical validation of a precision electromagnetic tremor measurement system in participants receiving deep brain stimulation for essential tremor”, Physiol. Meas. 2016; 37:1516-1527), noncontact measurements obtained from devices, such as Kinect (see, e.g., Sooklal S., et al., “Using the Kinect for detecting tremors: Challenges and opportunities; Proceedings of the IEEE-EMBS International Conference on Biomedical and Health Informatics (BHI); Valencia, Spain. 1-4 Jun. 2014; pp. 768-771), laser Doppler vibrometers (see, e.g., Sooklal S., et al., “Using the Kinect for detecting tremors: Challenges and opportunities; Proceedings of the IEEE-EMBS International Conference on Biomedical and Health Informatics (BHI); Valencia, Spain. 1-4 Jun. 2014; pp. 768-771), accelerometers or gyroscopes (see, e.g., Ossig A. A. C., et al., “Wearable sensor-based objective assessment of motor symptoms in Parkinson's disease”, J. Neural Transm. 2016; 123:57-64; and Rovini E., et al., “How Wearable Sensors Can Support Parkinson's Disease Diagnosis and Treatment: A Systematic Review”, Front. Neurosci. 2017; 11:555) and smart wearable technology.

Several scales have been established to as a means to rate the severity of PD, e.g., by measurement of motor manifestations, assessment of ability to perform daily functional activities, and symptomatic response to medication. The most common rating scales are the Unified Parkinson Disease Rating Scale (UPDRS), Hoehn and Yahr Staging Scale, and the Schwab and England Rating of Activities of Daily Living (see, e.g., Perlmutter J S, “Assessment of Parkinson disease manifestations”, Curr. Protoc Neurosci. 2009 October; Chapter 10: Unit10.1).

The Unified Parkinson Disease Rating Scale (UPDRS) is a tool that has gained greatest acceptance for evaluation of interventions and to follow patients (see, e.g., Martinez-Martin P, et al., “The Cooperative Multicentric Group Unified Parkinson's Disease Rating Scale characteristics and structure,” Mov. Disord. 1994; 9:76-8). A detailed description of the UPDRS is set forth in Perlmutter J S (“Assessment of Parkinson disease manifestations”, Curr Protoc Neurosci. 2009 October; Chapter 10: Unit10.1), the contents of which are expressly incorporate herein by reference.

The current UPDRS includes four subscales. Subscale 1 covers mentation, behavior, and mood. Subscale 2 rates activities of daily living. Subscale 3 is a clinician rating of the motor manifestations of PD. Subscale 4 covers complications of therapy. Data for subscales 1, 2, and 4 are elicited from patients and caregivers, whereas data for subscale 3 is examination-based. There are training tapes for the UPDRS subscales 2 and 3 and reviewing these can improve the reliability of the measures (see, e.g., Goetz C G, et al. “Standardized training tools for the UPDRS activities of daily living scale: Newly available teaching program,” Mov. Disord. 2003; 18:1455-1458); Goetz C G, et al., “Teaching tape for the motor section of the unified Parkinson's disease rating scale,” Mov. Disord. 1995; 10:263-266; and, e.g., Goetz C G et al., “Assuring interrater reliability for the UPDRS motor section: Utility of the UPDRS teaching tape, “Mov. Disord. 2004; 19:1453-1456). However, reliability of the other subscales depends on patient reporting in addition to examiner skills, but there is a training tape for the activities of daily living component subscale 2 (see, e.g., Louis E D, et al., “Reliability of patient completion of the historical section of the Unified Parkinson's Disease Rating Scale,” Mov. Disord. 1996; 11:185-192). The total UPDRS score and the UPDRS subscale scores are not interval scales, which means that there are not quantified, equal distances between values on these scales. For example, a score of 4 is greater than 2 but does not necessarily indicate twice the degree of severity. Each part of the rating is a rank order measure rather than a precise interval change. This must be considered when using these data for statistical analyses. Some sections of the UPDRS scale require multiple grades assigned to each extremity with a possible maximum of 199 points. A score of 199 on the UPDRS scale represents the worst (total disability) with a score of zero representing (no disability).

The Hoehn and Yahr scale was the first rating scale to describe the progression of PD and describes five stages to PD progression. It is a simple staging from 0 to 5 of the motor manifestations of PD, intended to reflect the degree of progression, and combines features of motor impairment and disability. However, the scale is not linear and may not even be rank order, with some people having greater disability with stage 2 (with substantial bradykinesia but good stability on the pull test) compared to some that have been ranked as stage 3 (that fall on the pull test but have relatively mild bradykinesia and rigidity). The rating was subsequently modified to include two half scores, as set forth below in Table 1 (see Goetz C G, et al., Movement Disorder Society Task Force report on the Hoehn and Yahr staging scale: Status and recommendations. Mov. Disord. 2004; 19:1020-1028).

TABLE 1
Modified Hoehn and Yahr Scale

Stage 0:
no signs of disease
Stage 1: unilateral disease (on one side)
In Stage 1, the earliest stage, the symptoms of PD are mild and only seen on one
side of the body (unilateral involvement), and there is usually minimal or no
functional impairment. The symptoms of PD at stage one may be so mild that the
person doesn't seek medical attention or the physician is unable to make a
diagnosis. Symptoms at stage one may include tremor, rigidity, or slowness of
movement in the arm or leg on one side of the body, or one side of the face may be
affected, impacting the expression.
Stage 1.5: unilateral disease plus axial involvement
Stage 2: bilateral disease, without impairment of balance
Stage two is still considered early disease in PD, and it is characterized by
symptoms on both sides of the body (bilateral involvement) or at the midline
without impairment to balance. Stage two may develop months or years after stage
one. Symptoms of PD in stage two may include the loss of facial expression on
both sides of the face and decreased blinking. Speech abnormalities may be present,
such as soft voice, monotone voice, fading volume after starting to speak loudly, or
slurring speech. There also may be stiffness or rigidity of the muscles in the trunk
that may result in neck or back pain, stooped posture, and general slowness in all
activities of daily living. Diagnosis may be easy at this stage if the patient has a
tremor, however, if stage one was missed and the only symptoms of stage two are
slowness or lack of spontaneous movement, PD could be misinterpreted as only
advancing age.
Stage 2.5: bilateral disease, with recovery on the pull test
Stage 3: mild to moderate bilateral disease; needs assistance to prevent falling on
pull test; physically independent
Stage three is considered mid-stage and is characterized by loss of balance and
slowness of movement. Balance is compromised by the inability to make the rapid,
automatic, and involuntary adjustments necessary to prevent falling, and falls are
common at this stage. All other symptoms of PD are also present at this stage, and
generally diagnosis is not in doubt at stage three. An important clarifying factor of
stage three is that the patient is still fully independent in their daily living activities,
such as dressing, hygiene, and eating.
Stage 4: severe disability, but still able to walk or stand unassisted
In stage four, PD has progressed to a severely disabling disease. Patients with stage
four PD may be able to walk and stand unassisted, but they are noticeably
incapacitated. Many use a walker to help them. At this stage, the patient is unable
to live an independent life and needs assistance with some activities of daily living.
Stage 5: wheelchair-bound or bedridden unless aided
Stage five is the most advanced and is characterized by confinement to a bed or
wheelchair. People with stage five PD may be unable to rise from a chair or get out
of bed without help, they may have a tendency to fall when standing or turning, and
they may freeze or stumble when walking. Around-the-clock assistance is required
at this stage to reduce the risk of falling and help the patient with all daily activities.
At stage five, the patient may also experience hallucinations or delusions. While the
symptoms worsen over time, it is worth noting that some patients with PD never
reach stage five. People with PD may also never experience some of the above
symptoms. In addition, there are treatments available that can help at every stage of
the disease.

The Schwab and England Scale is an “activities of daily living” (ADL) scale frequently used to provide a single estimate of a patient's ability to function. The rating is done by the person interviewing the patient and, frequently, a collateral source, such as a spouse. This rating varies from 0 to 100% using 5% increments and is set forth below in Table 2.

TABLE 2
The Schwab and England Scale

100%-completely independent; able to do all chores without slowness, difficulty,
or impairment; essentially normal; unaware of any difficulty
90%-completely independent and able to do all chores with some degree of
slowness, difficulty, or impairment; some activities might take twice as long;
beginning to be aware of difficulty
80%-completely independent in most chores; some activities take twice as long;
conscious of difficulty and slowness
70%-not completely independent; more difficulty with some chores; some tasks
now take three to four times as long; must spend a large part of the day with chores
60%-some dependency; can do most chores, but exceedingly slowly and with
much effort; some tasks cannot be done; common errors
50%-more dependent; needs help with about half of activities; slower and
experiencing difficulty with all tasks
40%-very dependent, but still able to assist with all chores; however, few can be
done independently
30%-all tasks require much effort; a few chores can be done alone or at least
started alone; much assistance needed
20%-no tasks done independently; patient can provide slight help with some
chores; but requires substantial assistance for all activities
10%-totally dependent and requires assistance with all activities of daily living
0%-vegetative functions with loss of control of swallowing, bladder, and bowel
functions; bedridden

### III. Doxycycline and Pharmaceutically Acceptable Salts Thereof

As used herein, the term Doxycycline (ADOXA®, ADOXA CK®, ADOXA PAK®, ADOXA TT®, DORYX®, MONODOX®, ORACEA®, PERIOSTAT®, VIBRAMYCIN CALCIUM®, VIBRAMYCIN HYCLATE®, AND VIBRA-TABS®) includes Doxycycline and pharmaceutically acceptable salts thereof. Doxycycline is a broad-spectrum antibiotic synthetically derived from oxytetracycline. It is on the World Health Organization's List of Essential Medicines and is available as a generic medicine.

Doxycycline belongs to the class of medicines known as tetracycline antibiotics. It is a second-generation tetracycline that was first discovered in 1967. Second-generation tetracyclines exhibit lesser toxicity than first-generation tetracyclines. Doxycycline is used to treat a wide variety of gram-positive and gram-negative bacterial infections.

Doxycycline is a broad-spectrum tetracycline class antibiotic used in the treatment of infections caused by bacteria and certain parasites. According to the National Library of Medicine of the National Institute of Health, doxycycline is also used to treat or prevent anthrax (a serious infection that may be spread on purpose as part of a bioterror attack) in people who may have been exposed to anthrax in the air and to treat plague and tuleramia (serious infections that may be spread on purpose as part of a bioterror attack). Doxycycline is also used to prevent malaria. Along with other medications, doxycycline is used to treat acne and rosacea (a skin disease that causes redness, flushing, and pimples on the face).

As it is a highly lipophilic drug, doxycycline crosses multiple membranes of target molecules. Doxycycline shows favorable intra-cellular penetration, with bacteriostatic activity against a wide range of bacteria. Doxycycline also exhibits antiparasitic properties and anti-inflammatory actions. Its anti-inflammatory effects were investigated in various inflammatory skin conditions, such as bullous dermatoses and rosacea.

Protein synthesis is essential for survival and functioning of cells, including bacteria. Doxycycline inhibits bacterial protein synthesis by allosterically binding to the 30S prokaryotic ribosomal subunit. The drug blocks the association charged aminoacyl-tRNA (aa-tRNA) with the ribosomal A site, which is the acceptor site on the mRNA-ribosome complex. Doxycycline ultimately impedes the elongation phase of protein synthesis and halts the production of essential proteins for bacterial survival and functioning.

Doxycycline mediates anti-inflammatory actions by preventing calcium-dependent microtubular assembly and lymphocytic proliferation, thereby inhibiting leukocyte movement during inflammation. It also inhibits nitric oxide synthase, which is an enzyme that produces nitric oxide, an inflammatory signaling molecule.

Doxycycline can be in a variety of forms. These forms include, e.g., liquid, semi solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Doxycycline intended for systemic or local delivery, for example, can be in the form of injectable or infusible solutions. Accordingly, doxycycline can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

In one embodiment, doxycycline is formulated as a powder for suspension. In another embodiment, doxycycline is formulated as a capsule (e.g., extended release). In another embodiment, doxycycline is formulated as a tablet (e.g., delayed release). In another embodiment, doxycycline is formulated as a syrup.

### IV. Levodopa

As used herein, the term Levodopa (DHIVY®, DUODOPAR, DUOPAR, INBRIJA®, PARCOPAR, PROLOPAR, RYTARY®, SINEMET®, STALEVOR, BIDOPAL®, DOPAR®, DOPARL®, DOPASOL®, AND DOPASTON®) includes Levodopa and pharmaceutically acceptable salts thereof. Levodopa is a dopamine precursor used in the management of PD, often in combination with carbidopa, as well as other related conditions. As levodopa can be metabolized to dopamine on either side of the blood-brain barrier, it is generally administered with a dopa decarboxylase inhibitor (e.g., carbidopa) to prevent metabolism until after it has crossed the blood-brain barrier (see, e.g., Djamshidian A, Poewe W, Parkinsonism Relat Disord. 2016 December; 33 Suppl 1: S9-S12). Once past the blood-brain barrier, levodopa is metabolized to dopamine and supplements the low endogenous levels of dopamine to treat symptoms of PD.

Levodopa is often viewed as the first-line drug for the management of PD motor symptoms. Levodopa is almost always given in combination with the drug carbidopa, which reduces or prevents the nausea that levodopa alone can cause. Carbidopa-levodopa is delivered in many forms, including immediate-release, controlled-release, or time-released oral tablets or capsules. In addition to pills and capsules, it is also available as an intestinal gel (DUOPAR) and inhaler (INBRIJAC®).

Carbidopa-levodopa immediate release tablets (SINEMET®) are available as 10-100 mg, 25-100 mg, or 25-250 mg doses. A typical treatment regimen is 100 to 1,000 mg of levodopa total per day (divided 3 to 4 times). In one embodiment, levodopa is administered at a dose of 100 mg once per day.

In one embodiment, levodopa is administered at a total dose of 100 to 1,000 mg of levodopa per day (e.g., administered as a divided dose, 3 to 4 times a day, as an immediate release tablet). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg. Carbidopa-levodopa controlled-release tablets (SINEMET CR*®) are available as 25-100 mg or 50-200 mg doses. A typical treatment regimen is 400 to 1,600 mg of levodopa in divided doses, depending on daily need.

In one embodiment, levodopa is administered at a total daily dose of 100 to 1,600 mg (e.g., administered in divided doses as a controlled-release tablet). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg. In one embodiment, levodopa is administered at a dose of 100 mg once per day.

Carbidopa-levodopa orally-disintegrating tablets (PARCOPA®) are available as 10-100 mg, 25-100 mg, or 25-250 mg doses. A typical treatment regimen is 100 to 1,000 mg of levodopa total per day (divided 3 to 4 times).

In one embodiment, levodopa is administered at a total daily dose of 100 to 1,000 mg (e.g., administered as a divided dose, 3 to 4 times a day, as an orally disintegrating tablet).

Carbidopa-levodopa enteral suspension (DUOPA®) is available as a 4.86/20 per mL dose. This form of carbidopa-levodopa is delivered through a surgically implanted tube in the small intestine, rather than a pill. Following surgery, this delivery method increases “on” time (when a patient has good symptom control and can move and function well) without troublesome, involuntary dyskinesia movements. A typical treatment regimen is up to 2,000 mg of levodopa over 16 hours.

In one embodiment, levodopa is administered at a dose of up to 2,000 mg over 16 hours (e.g., as an enteral suspension). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg. In one embodiment, levodopa is administered at a dose of 100 mg once per day.

Carbidopa-levodopa extended release (ER) capsules (RYTARY®) are available as 23.75-95 mg, 36.25-145 mg, 48.75-195 mg, or 61.25-245 mg doses. This form of carbidopa-levodopa contains beads of both medications that dissolve and are absorbed at different rates. This can improve “on” time while requiring fewer medication doses. Dosages of carbidopa-levodopa ER capsules are not interchangeable with dosages of other carbidopa-levodopa products. A typical treatment regimen is 855 to 2,340 mg of levodopa in divided doses, depending on daily need.

In one embodiment, levodopa is administered at a total daily dose of 855 to 2,340 mg of levodopa (e.g., administered in divided as an extended release capsule).

Carbidopa-levodopa entacapone tablets (STALEVO®) are available as 12.5-50-200 mg, 18.75-75-200 mg, 25-100-200 mg, 31.25-125-200 mg, or 37.5-150-200 mg, 50-200-200 mg doses. This is a combination drug which includes entacapone and carbidopa-levodopa in one pill. It is more convenient compared with carbidopa-levodopa and entacapone taken separately. A typical treatment regimen is 150 to 1,600 mg of levodopa total daily dose, depending on daily need (maximum 8 tablets daily).

In one embodiment, levodopa is administered at a total daily dose of 100 to 1,600 mg (e.g., administered as a tablet). For example, levodopa can be administered at a dose of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg, In one embodiment, levodopa is administered at a dose of 100 mg once per day.

Levodopa inhalation powder (INBRIJA®) is available as 42 mg or 84 mg doses. This is an inhaled form of levodopa most commonly used for early morning “off” times or sudden “offs” (e.g., PD motor and/or non-motor symptoms happen between medication doses), giving a low but quicker acting boost of dopamine. This is often in addition to a more stable regimen. The inhaled oral formulation of levodopa was approved by the U.S. Food and Drug Administration in 2018 to treat PD as adjunctive therapy to levodopa/carbidopa. Inhaled levodopa bypasses the intestinal absorption and hepatic metabolism of oral levodopa, and it is available in the form of dry powder (see, e.g., LeWitt P A, et al., Lancet Neurol. 2019 February; 18 (2): 145-154). A typical treatment regimen is 42 to 84 mg (1 to 2 capsules) up to 5 times per day.

In one embodiment, levodopa is administered at a dose of 42 to 84 mg, up to 5 times per day (e.g., as a capsule of inhalation powder). For example, levodopa can be administered at a dose of 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, or 84 mg. In one embodiment, levopoda is administered once a day, twice a day, three times a day, four times a day, or five times a day.

In one embodiment, levodopa is administered to the patient at subtherapeutic dose.

### V. Methods of Treatment

Provided herein are methods for treating a neurodegenerative disorder (e.g., PD) or symptom thereof (e.g., tremor) by administering doxycycline (or a pharmaceutically acceptable salt thereof) either alone or in combination with other therapeutic agents (e.g., levodopa) to a patient in need thereof.

As used herein, the term “subject” or “patient” is a human patient (e.g., a patient having a neurodegenerative disorder or symptom).

As used herein, the term “pediatric” patient is a human patient that has been classified by a physician or caretaker as belonging to a non-adult category and can include, e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such, e.g., one who is not a newborn, infant, child or adolescent, e.g., based on age, developmental status, physiological features, etc. Typically, adult patients are patients who are 18 years of age or older (≥18 years of age).

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one symptom of a neurodegenerative disorder.

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount clinically proven to alleviate at least one symptom of a neurodegenerative disorder. An effective amount can be administered in one or more administrations.

As used herein, the phrase “Optimal biologic dose (OBD)” is defined as the minimum dose of an agent or combination of agents that gives the most optimal and lasting in vivo response without clinically unacceptable toxicity. Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

As used herein, the terms “synergy”, “therapeutic synergy”, and “synergistic effect” refer to a phenomenon where treatment of patients with a combination of therapeutic agents (e.g., doxycycline and levodopa) manifests a therapeutically superior outcome to the outcome achieved by each individual constituent of the combination used when used alone (see, e.g., T. H. Corbett et al., 1982, Cancer Treatment Reports, 66, 1187). In this context a therapeutically superior outcome includes one or more of the following (a) an increase in therapeutic response that is greater than the sum of the separate effects of each agent alone at the same dose as in the combination; (b) a decrease in the dose of one or more agents in the combination without a decrease in therapeutic efficacy; (c) a decrease in the incidence of adverse events while receiving a therapeutic benefit that is equal to or greater than the monotherapy of each agent at the same dose as in the combination, (d) a reduction in dose-limiting toxicities while receiving a therapeutic benefit that is greater than the monotherapy of each agent; (e) a delay or minimization of the induction of drug resistance. Synergism of a drug combination can be determined, for example, according to the combination index (CI) theorem of Chou-Talalay (Chou et al., Adv. Enzyme Regul. 1984; 22:27-55; Chou, Cancer Res. 2010; 70 (2): 440-446).

“Relapse” or “recurrence” or “resurgence” are used interchangeably herein, and refer to the diagnosis of the return of signs and symptoms after a period of improvement or response.

As used herein, a “subtherapeutic dose” of a therapeutic compound (e.g., doxycycline or levodopa) refers to a dose which is lower than the usual/typical dose of said compound required to obtain a therapeutic effect.

The term “subtherapeutic amount” of a compound means an amount that would be below an accepted therapeutically effective amount. A subtherapeutic amount can be defined as an amount less than the FDA-approved dosage or dosages for a particular disease. Alternatively, considering that many drugs are used off-label, a subtherapeutic amount can be defined as an amount less than that typically prescribed by physicians for a particular disease. A sub-therapeutic amount may also take into account such factors as body mass, sex, age, renal or hepatic impairment, and other parameters which may affect the efficaciousness of a given amount of a particular drug. In some embodiments, a subtherapeutic amount may be 85% of a therapeutically effective amount. In other embodiments, a subtherapeutic amount may be 70% of a therapeutically effective amount. In other embodiments, a subtherapeutic amount may be 60% of a therapeutically effective amount. In other embodiments, a subtherapeutic amount may be 50% of a therapeutically effective amount. In other embodiments, a subtherapeutic amount may be 40% of a therapeutically effective amount. In other embodiments, a subtherapeutic amount may be 30% of a therapeutically effective amount. In further embodiments, it may be even less.

In other words, a subtherapeutic dose is an amount of a therapeutic compound (e.g., doxycycline or levodopa) which when administered to a patient, is not in itself sufficiently effective in the treatment of the claimed disorders (e.g., neurodegenerative disorder, such as PD). Yet, the combination is surprisingly sufficiently effective in the treatment of the claimed disorder, even if one or more of the therapeutic compounds (e.g., doxycycline or levodopa) are administered at doses which are not sufficiently effective in the treatment of the disorder.

In one embodiment, a method of treating a neurodegenerative disorder or a symptom thereof in a patient is provided, the method comprising administering to the patient a therapeutically active amount of doxycycline (or a pharmaceutically acceptable salt thereof).

In one embodiment, a method of treating PD or a symptom thereof (e.g., tremor) in a patient is provided, the method comprising administering to the patient a therapeutically active amount of doxycycline (or a pharmaceutically acceptable salt thereof).

In one embodiment, a method of treating a neurodegenerative disorder or a symptom thereof in a patient is provided, the method comprising administering to the patient a therapeutically active amount of doxycycline (or a pharmaceutically acceptable salt thereof) in combination with a therapeutically active amount of levodopa (or a pharmaceutically acceptable salt thereof).

In one embodiment, a method of treating PD or a symptom thereof (e.g., tremor) in a patient is provided, the method comprising administering to the patient a therapeutically active amount of doxycycline (or a pharmaceutically acceptable salt thereof) in combination with a therapeutically active amount of levodopa (or a pharmaceutically acceptable salt thereof).

In one embodiment, a method of treating a neurodegenerative disorder or a symptom thereof in a patient is provided, the method comprising administering to the patient a therapeutically active amount of doxycycline (or a pharmaceutically acceptable salt thereof) in combination with a therapeutically active amount of levodopa (or a pharmaceutically acceptable salt thereof), wherein administration of doxycycline and levodopa in combination results in a synergistic therapeutic effect compared to administration of either doxycycline or levodopa alone.

In one embodiment, a method of treating PD or a symptom thereof (e.g., tremor) in a patient is provided, the method comprising administering to the patient a therapeutically active amount of doxycycline (or a pharmaceutically acceptable salt thereof) in combination with a therapeutically active amount of levodopa (or a pharmaceutically acceptable salt thereof), wherein administration of doxycycline and levodopa in combination results in a synergistic therapeutic effect compared to administration of either doxycycline or levodopa alone.

In one embodiment, a method of treating a neurodegenerative disorder or a symptom thereof in a patient is provided, the method comprising administering doxycycline (or a pharmaceutically acceptable salt thereof) in combination with levodopa, wherein what would normally be a subtherapeutic dose of doxycycline or levodopa alone, is rendered effective by the combination. Thus, a dose of doxycycline below the standard therapeutic dose and/or a dose of levodopa below the standard therapeutic dose, which would ordinarily be without therapeutic effect in humans, is found to have therapeutic effective when doxycycline and levodopa are administered in combination. In other words, by administering a combination of doxycycline and levodopa, at least one or both of doxycycline and/or levodopa can be at administered at a subtherapeutic dose, while still resulting in a similar efficacy.

In one embodiment, a method of treating PD or a symptom thereof (e.g., tremor) in a patient is provided, the method comprising administering doxycycline (or a pharmaceutically acceptable salt thereof) in combination with levodopa, wherein what would normally be a subtherapeutic dose of doxycycline or levodopa alone, is rendered effective by the combination. Thus, a dose of doxycycline below the standard therapeutic dose and/or a dose of levodopa below the standard therapeutic dose, which would ordinarily be without therapeutic effect in humans, is found to have therapeutic effective when doxycycline and levodopa are administered in combination. In other words, by administering a combination of doxycycline and levodopa, at least one or both of doxycycline and/or levodopa can be at administered at a subtherapeutic dose, while still resulting in a similar efficacy.

In one embodiment, a method of treating a neurodegenerative disorder or a symptom thereof in a patient is provided, the method comprising administering to the patient doxycycline (or a pharmaceutically acceptable salt thereof) in combination with levodopa (or a pharmaceutically acceptable salt thereof), wherein at least one of doxycycline and/or levodopa is administered at a subtherapeutic dose. In some embodiments, the subtherapeutic dose of doxycycline and/or subtherapeutic dose of levodopa is a dose which is lower than the dose of doxycycline and/or the dose of levodopa, required to obtain a therapeutic effect in the patient, when administered alone.

In one embodiment, a method of treating PD or a symptom thereof (e.g., tremor) in a patient is provided, the method comprising administering to the patient doxycycline (or a pharmaceutically acceptable salt thereof) in combination with levodopa (or a pharmaceutically acceptable salt thereof), wherein at least one of doxycycline and/or levodopa is administered at a subtherapeutic dose. In some embodiments, the subtherapeutic dose of doxycycline and/or subtherapeutic dose of levodopa is a dose which is lower than the dose of doxycycline and/or the dose of levodopa, required to obtain a therapeutic effect in the patient, when administered alone.

### VI. Other Combination Treatments

Doxycycline (or a pharmaceutically acceptable salt thereof) can be administered according to the methods described herein: (1) alone, (2) in combination with levodopa (or a pharmaceutically acceptable salt thereof), (3) in combination with one or more other therapeutic agents, or (4) in combination with levodopa and one or more other therapeutic agents. Any other suitable therapeutic agent can be administered.

In one embodiment, the other therapeutic agent is a Kv7 channel activator (e.g., retigabine, flupirtine, or endocannabinoid-like molecules).

In another embodiment, the other therapeutic agent is an antibody to alpha-synuclein (e.g., Abeam's “Syn204”, Alexis's “15G 7”, Becton Dickinson's “42”, Chemicon's “7B 2, 12”, NovaCastra's “KM51”, SantaCruz's “Syn211”, or Zymed's “LB509”).

In another embodiment, the other therapeutic agent is a decarboxylase inhibitor, such as carbidopa.

In another embodiment, the other therapeutic agent is a dopamine agonist (e.g., Parlodel (BROMOCRIPTINE®), Cabergoline, Apokyn (APOMORPHINE®), Mirapex (PRAMIPEXOLE®), Requip (ROPINIROLE®), or Neupro (ROTIGOTINE®).

In another embodiment, the other therapeutic agent is a monoamine oxidase (MAO) B inhibitor (e.g., socarboxazid (MARPLAN®), Phenelzine (NARDIL®), Selegiline (EMSAM®), Tranylcypromine (PARNATE®), selegiline hydrochloride (ELDEPRYL®), selegiline hydrochloride (ZELAPAR®), rasagiline (AZILECT®) or safinamide (XADAGO®).

In another embodiment, the other therapeutic agent is a catechol O-methyltransferase (COMT) inhibitor (e.g., entacapone (COMTAN®, COMTESS®, STALEVO®), nebicapone, nitecapone, opicapone (ONGENTYS®), or tolcapone (TASMAR®).

In another embodiment, the other therapeutic agent is an anti-cholinergic agent (e.g., (1) an antimuscarinic agent, such as an antipsychotic (CLOZAPINE®, QUETIAPINE®), atropine, benztropine (COGENTIN®), biperiden, chlorpheniramine, certain SSRIs (PAROXETINE®), dicyclomine (DICYCLOVERINE®), dimenhydrinate, diphenhydramine, doxepin, doxylamine, flavoxate, glycopyrronium/-late, hyoscyamine, ipratropium, orphenadrine, oxitropium, oxybutynin, promethazine, propantheline bromide, scopolamine, solifenacin, tolterodine, tiotropium, tricyclic antidepressants, trihexyphenidyl, amantadine, tropicamide, and umeclidinium, or (2) an antinicotinic agent, such as bupropion, dextromethorphan, doxacurium, dexamethonium, mecamylamine, and tubocurarine).

In another embodiment, the other therapeutic agent is an adenosine receptor antagonist (A2A receptor antagonist) (e.g., caffeine, theophylline or istradefylline (NOURIANZ®), or Nuplazid (PIMAVANSERIN®)).

In another embodiment, the other therapeutic agent is a TNF-alpha targeting agent or inhibitor (e.g., liximab (REMICADE®), adalimumab (HUMIRA®), certolizumab pegol (CIMZIA®), golimumab (SIMPONI®), and etanercept (ENBREL®).

In another embodiment, the other therapeutic agent is a IL17A targeting agent or inhibitor (e.g., Secukinumab (COSENTYX®), Ixekizumab (TALTZ®), Bimekizumab (BIMZELX®), or Brodalumab (SILIQ®).

In another embodiment, the other therapeutic agent is deep brain stimulation.

### VII. Outcomes

Provided herein are methods for treating a neurodegenerative disorder (e.g., PD) or symptom in a patient comprising administering to the patient doxycycline (or a pharmaceutically acceptable salt thereof) either alone or in combination with other therapeutic agents (e.g., levodopa). Patients treated according to the methods disclosed herein experience improvement in at least one symptom of a neurodegenerative disorder.

Exemplary symptoms of neurodegenerative disorders include, but are not limited to abnormal mobility, abnormal balance, abnormal movements (e.g., tremor), abnormal swallowing, abnormal bladder and bowel function (e.g., irritable bowel, belly pain, belly discomfort, diarrhea, abdominal bloating, abdominal gas, abdominal bloating, and/or constipation), blood pressure fluctuation, abnormal sleep, abnormal breathing (e.g., shortness of breath), abnormal heart function (e.g., heart palpitations), abnormal memory, abnormal cognitive abilities, abnormal mood, abnormal speech, anxiety, depression, stress, fatigue, feelings of panic, fear, uneasiness, problems in sleeping, cold or sweaty hands and/or feet, mood liability, mania, impaired concentration or attention, cognitive problems, obsessions, compulsions, repetitive behaviors, aggression, social phobias or impairments, stage fright, an inability to be still and calm, dry mouth, numbness or tingling in the hands or feet, nausea, muscle tension, dizziness apathy, elation, disinhibition, irritability, wandering, and/or combinations thereof.

In one embodiment, the patient is a PD patient. Exemplary symptoms of PD include, but are not limited to, tremor, slowed movement (bradykinesia), rigid muscles, impaired posture and balance, loss of automatic movements, speech changes, and writing changes. Accordingly, in some embodiments, a PD patient treated according to the methods described herein experiences an improvement in one or more of these symptoms.

In another embodiment, the patient is a PD patient and the treatment results in an improvement according to a recognized clinical scale for evaluating PD. For example, in one embodiment, the treatment results in an improvement according to the Unified Parkinson Disease Rating Scale (UPDRS). A score of 199 on the UPDRS scale represents the worst (total disability) with a score of zero representing (no disability). Accordingly, in one embodiment, the treatment results in a decrease in score compared to baseline by, for example, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 199 points. In another embodiment, the treatment results in a 5, 10, 15, 20, 25, 30, 35, 40, 45, 50, 55, 60, 65, 70, 75, 80, 85, 90, 95, 100% decrease in score compared to baseline.

In another embodiment, the patient is a PD patient and the treatment results in an improvement according to the Hoehn and Yahr Staging Scale. The Hoehn and Yahr Staging Scale includes 7 different stages ranging from no disease (Stage 1) to wheelchair bound or bedridden (Stage 5), i.e., Stage 0, Stage 1, Stage 1.5, Stage 2, Stage 2, Stage 2.5, Stage 3, Stage 4, and Stage 5. In one embodiment, the treatment results in a one or more stage decrease in score compared to baseline (e.g., a one, two, three, four, five, six, seven, or eight stage decrease).

In another embodiment, the patient is a PD patient and the treatment results in an improvement according to the Schwab and England Rating of Activities of Daily Living. This rating varies from 0 (completely independent) to 100% (vegetative functions), using 10% increments and includes 11 possible ratings, i.e., 0%, 10%, 20%, 30%, 40%, 50%, 60%, 70%, 80%, 90%, and 100%. In one embodiment, the treatment results in a one or more rating decrease compared to baseline (e.g., a one, two, three, four, five, six, seven, eight, one or ten rating decrease).

In some embodiments, the patient is a PD patient and the treatment results in an improvement in tremor (e.g., a reduction in tremor), for example, by about 5%, 10%, 15%, 20%, 25%, 30%, 35%, 40%, 45%, 50%, 55%, 60%, 65%, 70%, 75%, 80%, 85%, 90%, 95%, or 100%). The improvement can be assessed by any suitable means, including, but not limited to electromyography (EMG), an electromagnetic motion tracker, noncontact measurements obtained from devices (e.g., Kinect), a laser Doppler vibrometer, an accelerometer, a gyroscope, or a smart wearable technology device.

### VIII. Kits and Unit Dosage Forms

Also provided herein are kits that include a dose of doxycycline adapted for use in the preceding methods. In some embodiments, the kits further comprise a dose of levodopa. The kits optionally also can include instructions, e.g., comprising administration schedules, to allow a practitioner (e.g., a physician, nurse, or patient) to administer the composition contained therein to administer the composition to a patient having a neurodegenerative disorder, such as PD. The kit also can include a syringe.

In one embodiment, the kit comprises: (a) a dose of doxycycline and (b) instructions for using doxycycline in the methods described herein.

In another embodiment, the kit comprises (a) a dose of doxycycline (b) a dose of levodopa and (c) instructions for using doxycycline and levodopa in the methods described herein. In one embodiment, the dose of doxycycline is a therapeutically effective amount. In one embodiment, the dose of levodopa is a therapeutically effective amount. In one embodiment, the dose of doxycycline and/or levodopa is a subtherapeutic dose.

### IX. Uses

Provided herein are uses of doxycycline for treatment of a neurodegenerative disorder (e.g., PD) or symptom (e.g., tremor), wherein doxycycline (or a pharmaceutically acceptable salt thereof) is administered to the patient in an amount and with a frequency to treat the neurodegenerative disorder or symptom.

Further provided are uses of doxycycline in combination with levodopa (or a pharmaceutically acceptable salt thereof) for treatment of a neurodegenerative disorder (e.g., PD) or symptom (e.g., tremor), wherein doxycycline and levodopa are administered to the patient in an amount and with a frequency to treat the neurodegenerative disorder or symptom. In one embodiment, the combination of doxycycline and levodopa results in a synergistic therapeutic effect compared to administration of either doxycycline or levodopa alone. In another embodiment, the combination of doxycycline and levodopa allows for doxycycline and/or levodopa to be administered at a subtherapeutic dose.

The following example is merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### Example

Patient A is a 59 year-old diagnosed with PD approximately one and a half years earlier and treated with levodopa. In addition to typical symptoms of tremor, the patient also exhibited seborrheic dermatitis, which is an inflammatory skin condition known to be associated with a higher incidence in Parkinson's disease. The patient also experienced intermittent irritable bowel disorder symptoms. Prior to initiation of doxycycline, the patient required dose increases of levodopa every approximate 3 months as worsening PD, most notably tremor. The patient was initiated on doxycycline 100 mg tablet once a day for their skin symptoms and within 3 weeks had resolution of seborrheic dermatitis. Quite surprisingly and unexpected, the patient also demonstrated a clinically significant reduction in tremor after starting the doxycycline. After approximately 8 weeks, the patient discontinued doxycycline and the seborrheic dermatitis recurred within 2 weeks with a clinically worsened tremor. The patient restarted doxycycline and again experienced a remission of seborrheic dermatitis and improvement in tremor. The patient's inflammatory skin condition again went into remission and Parkinson's tremor clinically improved. The patient stayed on doxycycline for two additional months but then stopped the doxycycline due to nausea. Once again upon doxycycline de-challenge, the seborrheic dermatitis returned within one month and tremor clinically worsened. The patient restarted doxycycline at 50 mg and demonstrated partial improvement in skin symptoms and PD. Dose was again increased to 100 mg with remission of seborrheic dermatitis and improvement in tremor. The patient remained on doxycycline 100 mg tablet once daily for approximately two years and experienced complete remission of seborrheic dermatitis, absence of irritable bowel symptoms and clinically significant improvement in tremor. In addition to the improvement in PD tremor, the patient for the first time did not require any increase in the Parkinson's treatment and dose of levodopa remained stable for approximately two years after starting treatment with doxycycline 100 mg.

1. A method of treating a neurodegenerative disorder or symptom, comprising administering to a patient in need thereof a therapeutically active amount of doxycycline.

2. The method of claim 1, wherein the neurodegenerative disorder is selected from the group consisting of amyotrophic lateral sclerosis (ALS), Huntington's disease, Parkinson's Disease (PD), supranuclear palsy, frontotemporal dementia, frontotemporal lobar degeneration, frontotemporal neurocognitive disorder, Alzheimer's disease, mild cognitive impairment, mild cognitive impairment due to Alzheimer's disease, ataxia, motor neuron disease, multiple system atrophy, hereditary ataxia, dementia, memory loss, mental retardation, Rett Syndrome, progressive supranuclear palsy, Creutzfeldt-Jakob disease, neurofibromatosis, brain injury, stroke, multiple sclerosis, and α-synucleinopathy (e.g., Lewy body disease).

3. (canceled)

4. The method of claim 1, wherein the symptom is selected from the group consisting of abnormal mobility, abnormal balance, abnormal movements, abnormal swallowing, abnormal bladder and bowel function (e.g., irritable bowel, belly pain, belly discomfort, diarrhea, abdominal bloating, abdominal gas, abdominal bloating, and/or constipation), blood pressure fluctuation, abnormal sleep, abnormal breathing (e.g., shortness of breath), abnormal heart function (e.g., heart palpitations), abnormal memory, abnormal cognitive abilities, abnormal mood, abnormal speech, anxiety, depression, stress, fatigue, feelings of panic, fear, uneasiness, problems in sleeping, cold or sweaty hands and/or feet, mood liability, mania, impaired concentration or attention, cognitive problems, obsessions, compulsions, repetitive behaviors, aggression, social phobias or impairments, stage fright, an inability to be still and calm, dry mouth, numbness or tingling in the hands or feet, nausea, muscle tension, dizziness apathy, elation, disinhibition, irritability, wandering, or combinations thereof.

5. The method of claim 1, wherein the therapeutically active amount of doxycycline is between 50 mg to 200 mg once a day.

6. The method of claim 1, wherein the therapeutically active amount of doxycycline is 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg once a day.

7-8. (canceled)

9. The method of claim 1, wherein the patient was previously administered levodopa.

10. The method of claim 1, wherein the method further comprises administering to the patient levodopa.

11-14. (canceled)

15. The method of claim 10, wherein levodopa is administered at a dose of between about 100 mg to 2,340 mg once a day.

16. The method of claim 10, wherein levodopa is administered at a dose of between about 100 mg once a day.

17. The method of claim 10, wherein administration of doxycycline and levodopa results in a synergistic therapeutic effect compared to administration of either doxycycline or levodopa alone.

18. The method of claim 10, wherein doxycycline and levodopa are administered in combination and wherein at least one of doxycycline and/or levodopa are administered at a subtherapeutic dose.

19. The method of claim 18, with the subtherapeutic dose of doxycycline and/or subtherapeutic dose of levodopa is a dose which is lower than the dose of doxycycline and/or the dose of levodopa, required to obtain a therapeutic effect in the patient, when administered alone.

20. The method of claim 1, wherein the neurodegenerative disorder is PD and the treatment results in an improvement according to the Unified Parkinson Disease Rating Scale (UPDRS), the Hoehn and Yahr Staging Scale, and/or the Schwab and England Rating of Activities of Daily Living.

21. The method of claim 1, wherein the neurodegenerative disorder is PD and the treatment results in an improvement in tremor.

22. (canceled)

23. The method of claim 1, wherein doxycycline is administered in a combination with an additional therapeutic agent.

24. The method of claim 23, wherein the additional therapeutic agent is a Kv7 channel activator, an antibody to alpha-synuclein, carbidopa, a dopamine agonist, a monoamine oxidase (MAO) B inhibitor, a catechol O-methyltransferase (COMT) inhibitor, an anti-cholinergic agent, and an adenosine receptor antagonist (A2A receptor antagonist), or a combination thereof.

25. The method of claim 23, wherein the additional therapeutic agent is a TNF-alpha targeting agent, a IL17A targeting agent, or a combination thereof.

26. The method of claim 1, wherein doxycycline is administered in a combination with deep brain stimulation.

27. A kit comprising: (a) a dose of doxycycline and (b) instructions for using the doxycycline in the method of claim 1.

28. A kit comprising: (a) a dose of doxycycline (b) a dose of levodopa and (c) instructions for using doxycycline and levodopa in the method of claim 10.

29-33. (canceled)
